(19) United States
(12) Patent Application Publication
(10) Pub. No.: US 2025/0114424 A1
(43) Pub. Date: Apr. 10, 2025

(54) PLANT EXTRACT-BASED COMPOSITION AND ITS USE FOR TREATING GLUCOSE METABOLISM DISORDERS

(71) Applicant: ESSERRE PHARMA SRL, Roma (RM) (IT)

(72) Inventors: Costanza Valentina RICCIONI, Roma (RM) (IT); Amedeo SQUILLACE GRECO, Roma (RM) (IT)

(73) Assignee: ESSERRE PHARMA SRL, Roma (RM) (IT)

(21) Appl. No.: 18/291,306

(22) PCT Filed: Aug. 4, 2022
(86) PCT No.: PCT/EP2022/071975

§ 371 Date: Jan. 23, 2024

(30) Foreign Application Priority Data
Aug. 4, 2021 (IT) 102021000021170

(51) Int. Cl. A61K 36/752; A61K 31/353; A61K 31/7048; A61K 33/24; A61P 3/08

(52) U.S. Cl. CPC A61K 36/752; A61K 31/353; A61K 31/7048; A61K 33/24; A61P 3/08

(57) ABSTRACT

The present invention relates to a composition containing Citrus sinensis (L.) Osbeck fruit and lemon extracts and preferably a trace element, which is preferably a mineral such as Chromium, Calcium, Magnesium, Iron, Copper, Zinc, Iodine, Manganese, Potassium, Sodium, Selenium, Molybdenum, Fluoride, Chloride or Phosphorus or combinations thereof, and their salts and/or a vitamin such as A, D, E, K, B1, B2, C, Niacin, Pantothenic acid, B6, Folic acid, B12 or Biotin or combinations thereof.

### FIELD OF THE INVENTION

The present invention relates to a composition based on several plant extracts having particular characteristics, and their use in the pharmaceutical field or as a food supplement in the prevention and treatment of risk factors related to alterations in glucose metabolism. More in particular, the present composition relates to a composition comprising: a complex of citrus extracts (such as blood orange and lemon) and possibly a trace element (such as Vitamin and/or Mineral, for example chromium).

### PRIOR ART

Cardiovascular diseases (diabetes mellitus, stroke, heart attack, atherosclerosis, etc.) are the leading cause of morbidity throughout the world, particularly in Western and more “developed” nations, but with an increasing number of cases in developing countries as well. Type II diabetes mellitus is a clinical condition characterized by a combination of impaired insulin secretion and peripheral insulin resistance. The consequence of a lack of compensation with increased insulin production by the beta cells of the pancreas is an increase in blood glucose that can lead to the development of complications such as retinopathy, nephropathy, neuropathy and atherosclerosis.

Any condition characterized by fasting blood glucose values in venous plasma equal to or greater than 126 mg/dL or blood glucose values 2 hours after a 75 g glucose load equal to or greater than 200 mg/dL and/or glycated haemoglobin (HbAlc) values equal to or greater than 6.5% is considered diabetes.

To date, subclinical conditions of glucose metabolism alterations are very common and, if not treated early, can evolve into chronic pathological conditions.

Prediabetes is a sub-clinical condition in which fasting blood glucose, blood glucose after glucose loading and/or glycated haemoglobin are higher than normal but lower than those attributable to the diagnosis of diabetes.

Impaired fasting glycaemia (IFG) is characterized by fasting blood glucose values between 100 and 125 mg/dL.

The presence of HbAlc values between 5.7 and 6.4% is also considered prediabetes, as well as the presence of blood glucose values 2 hours after a 75 g glucose load which are between 140 and 199 mg/dL is a condition defined as “impaired glucose tolerance” (IGT).

It is estimated that approximately 70% of individuals with prediabetes will develop diabetes during their lifetime.

There are about 463 million people worldwide with diabetes mellitus, 1 in 11 people between the ages of 20 and 79 and 374 million people with impaired glucose tolerance (IGT) or 1 in 13 people (20-79 years).

It is estimated that in 2030 there will be 578 million adults with diabetes and by 2045 these numbers will reach 700 million. (IDF Atlas 9th edition, 2019).

The drug of choice in the treatment of diabetes is metformin (biguanide class), which improves the sensitivity of tissues to insulin. The mechanism of action of metformin seems to comprise the activation of AMPK (activated protein kinase adenosine monophosphate) which plays a fundamental role in the body's energy balance by regulating carbohydrate and lipid metabolism.

Side effects of metformin are quite common and include nausea, vomiting, diarrhoea, constipation, flatulence and, more rarely, lactic acidosis.

Other oral antidiabetics used in the treatment of type II diabetes include sulfonylureas, glinides, glitazones, DPP-4 inhibitors, intestinal alpha-glucosidase inhibitors, SGLT-2 renal glucose transporter inhibitors.

All of these drugs have almost the same side effects as metformin, some of which may also cause weight gain. Considering the multifactorial etiology of cardiovascular diseases, many patients require polytherapy, often consisting of antihypertensive, antidiabetic, and lipid-lowering drugs. This can more often lead to the onset of side effects and the abandonment of therapies.

However, scientific evidence shows that the first choice treatment in the prevention and treatment of prediabetes and diabetes is lifestyle modification, i.e., balanced nutrition and regular physical activity.

One study (Knowler W C, Barrett-Connor E, Fowler S E, Hamman R F, Lachin J M, Walker E A, Nathan D M; Diabetes Prevention Program Research Group. Reduction in the incidence of type 2 diabetes with lifestyle intervention or metformin. N Engl J Med. 2002 Feb. 7; 346 (6): 393-403. doi: 10.1056/NEJMoa012512. PMID: 11832527; PMCID: PMC1370926.) demonstrated that pharmacological treatment achieved lower results than lifestyle changes (31% vs 58% reduction in the incidence of diabetes).

Despite the obvious benefit of adopting healthy lifestyles in the prevention and treatment of diabetes, there are numerous subjective and objective obstacles in adopting healthy lifestyles and drug therapy is often undertaken following the failure of the first approach.

The use of so-called “nutraceuticals”, foods or substances of plant or natural origin which are concentrated sources of nutrients or promote the physiological functions of the body can be a useful tool, as part of a proper lifestyle.

Nutraceuticals can play a role in reducing disease risk factors, helping to improve the therapeutic response of patients, also potentially delaying or cancelling the need for the use of pharmacological therapies.

Considering the poor compliance with the diet on the one hand and the side effects related to pharmacological therapy on the other, it is clear that it is necessary to identify new compositions of natural origin, characterized by a high safety of use and tolerability, which have a wide spectrum of action and are effective in the reduction of cardio-metabolic risk factors, for all those individuals who cannot or do not want to follow a pharmacological therapy, as in the cases of subjects in primary prevention, subjects with prediabetes or those intolerant to the pharmacological therapies of choice, young people, patients in pharmacological polytherapy, for whom a higher level of alertness is necessary for possible pharmacokinetic and pharmacodynamic interactions.

### Blood and Blonde Orange

The blood orange is a fruit of the plants of Citrus sinensis (L.) Osbeck, family Rutaceae, and characteristic of the varieties of Moro, Tarocco and Sanguinello orange, whose cultivation is widespread in the areas of the Mediterranean basin and in particular in Sicily.

It is distinguished by the red colour of the pulp, conferred by the content of anthocyanins, compounds of the flavonoid family.

Blonde orange as well is a fruit of the plants of Citrus Sinensis (L.) Osbeck, family Rutaceae with a yellow-orange pulp and juice and the varieties most widespread are Navelin, Washington, Valencia, Navel.

The fruit of Citrus sinensis (L.) Osbeck is rich in flavonoids, especially Hesperidin, Diosmin, Naringin, Narirutin, Didymin, as well as other phenolic compounds such as anthocyanins and hydroxycinnamic acids.

Epidemiological studies and meta-analyses have associated the intake of citrus fruits rich in flavonoids with a lower risk of heart attack (Cassidy, A. et al. “Dietary Flavonoids and Risk of Stroke in Women.” Stroke 43 (2012): 946-951) and with reduced incidence of certain cancers (Mccullough M L., Et al. “A prospective study of diet and stomach cancer mortality in United States men and women.” Cancer Epidemiol Biomarkers Prev. (2001) November; 10(11):1201-5).

Several studies have demonstrated the antioxidant properties of orange polyphenols. Hesperidin, Diosmin and Troxerutin are widely used in the treatment of venous insufficiency (Ulloa, J. H. Micronized Purified Flavonoid Fraction (MPFF) for Patients Suffering from Chronic Venous Disease: A Review of New Evidence. Adv Ther 36, 20-25 (2019). https://doi.org/10.1007/s12325-019-0884-4).

Hesperidin (3′,5,7-trihydroxy-4′-methoxy-flavanone-7-rhamnoglucoside) is the most present flavonoid in Citrus Sinensis L. Osbeck extract and exerts biological activity (also through its metabolite hesperitin). The molecular mechanisms underlying the physiological effects of hesperidin and hesperetin are not yet known.

In a preclinical study in rats with streptozocin-induced diabetes, the administration of 40 mg/kg of hesperidin for 30 days reduced fasting glycaemia, insulinemia and glycated haemoglobin (Sundaram R, Nandhakumar E, Haseena Banu H. Hesperidin, a citrus flavonoid ameliorates hyperglycemia by regulating key enzymes of carbohydrate metabolism in streptozotocin-induced diabetic rats. Toxicology Mechanisms and Methods. 2019 November; 29(9):644-653.) A recent study (Martínez-Noguera, F. J., Marín-Pagán, C., Carlos-Vivas, J., Rubio-Arias, J. A., & Alcaraz, P. E. (2019). Acute Effects of Hesperidin in Oxidant/Antioxidant State Markers and Performance in Amateur Cyclists. Nutrients, 11(8), 1898) showed that a single dose of 500 mg of hesperidin improved physical performance and modulated oxidative stress in a group of semi-professional cyclists.

Another study showed that the administration of 500 mg of hesperidin daily for 6 weeks reduced inflammation and blood pressure in subjects with type II diabetes mellitus (Homayouni, F., Haidari, F., Hedayati, M., Zakerkish, M., & Ahmadi, K. (2018). Blood pressure lowering and anti-inflammatory effects of hesperidin in type 2 diabetes; a randomized double-blind controlled clinical trial. Phytotherapy research: PTR, 32(6), 1073-1079).

### Lemon

Lemon (Citrus limon (L.) Osbeck) is a plant in the citrus genus of the family Rutaceae. The lemon fruit is rich in polyphenols such as hydroxycinnamic acids and flavonoids. Among these, the ones that most characterize lemon are eriocitrin, naringin, hesperidin, narirutin. A study on an eriocitrin-rich lemon extract reported an improvement in metabolic parameters in individuals with prediabetes after consuming 200-800 mg per day of eriocitrin for 3 months (Ribeiro, C. B., Ramos, F. M., Manthey, J. A., & Cesar, T. B. (2019). Effectiveness of Eriomin® in managing hyperglycemia and reversal of prediabetes condition: A double-blind, randomized, controlled study. Phytotherapy research: PTR, 33(7), 1921-1933)

Avila-Gálvez and colleagues conducted a pharmacokinetic study comparing the intake of an orange extract consisting mainly of hesperidin (260 mg) and a lemon extract rich in flavonoids (744 mg of which 260 mg of eriocitrin) in 16 healthy volunteers of both sexes. The study showed that taking lemon extract provides a higher amount of circulating phase II flavanone metabolites with respect to taking hesperidin. However, no significant beneficial effects were reported on a number of biomarkers linked to metabolism, inflammation and oxidative stress, measured after a high-fat and high-sugar meal (Ávila-Gálvez, M. Á., Giménez-Bastida, J. A., González-Sarrías, A., & Espín, J. C. (2021). New Insights into the Metabolism of the Flavanones Eriocitrin and Hesperidin: A Comparative Human Pharmacokinetic Study. Antioxidants (Basel, Switzerland), 10(3), 435.). The results of the studies focused on cardiovascular risk factors conducted with citrus flavonoids so far are sometimes inconsistent both due to the variability of the inter-individual response to flavonoid intake and to the low bioavailability of some flavonoids, such as hesperidin, characterized by poor water solubility. (Demonty, I., Lin, Y., Zebregs, Y. E., Vermeer, M. A., van der Knaap, H. C., Jäkel, M., & Trautwein, E. A. (2010). The citrus flavonoids hesperidin and naringin do not affect serum cholesterol in moderately hypercholesterolemic men and women. The Journal of nutrition, 140(9), 1615-1620.)

There is still a need for a composition for treating diseases or disorders associated with alterations in glucose and glyco-lipid metabolism without presenting side effects.

### DESCRIPTION OF THE INVENTION

The present invention therefore relates to a composition of naturally occurring plant extracts characterized by a favourable profile of safety and efficacy in reducing risk factors related to the development of chronic cardio-metabolic diseases.

The object of the present invention is a composition for pharmaceutical use or as a nutritional supplement comprising a mixture of Citrus sinensis (L.) Osbeck fruit, such as blood orange, and lemon extracts, preferably characterized by a minimum flavonoid content equal to 48% by weight on the total weight of the composition and a content equal to 0.8% w/w in hydroxycinnamic acids. The flavonoids constitute the composition according to the following percentages by weight on the total weight of the composition: hesperidin 34%, eriocitrin 7%, anthocyanins 0.2%, other flavonoids including naringin, neoeriocitrin, neoesperidin, didymin, narirutin 7%.

The composition according to the invention was effective in reducing some cardio-metabolic risk factors, typical of individuals with impaired glucose tolerance or with impaired fasting glycaemia, such as subjects with type II diabetes mellitus, prediabetes or metabolic syndrome.

In fact, the composition of the present invention has surprisingly allowed to obtain statistically significant reductions in subjects treated with said composition with respect to both the baseline and the placebo group of the following parameters: Glycated haemoglobin (HbAlc), fasting plasma glucose (FPG), fasting insulinemia (FPI), HOMA-index, triglycerides.

The reduction of these parameters demonstrates the efficacy of the present composition in the prevention and/or treatment of subclinical conditions of impaired glucose metabolism which, if not treated early, can evolve into chronic pathological conditions. Therefore the present composition is effective in the prevention and/or treatment of alterations in glucose and/or glycolipid metabolism and/or a metabolic disorder, for example type II diabetes mellitus, prediabetes or metabolic syndrome, obesity, dyslipidaemia and/or hyperglycemia and/or for use in reducing the cardio-metabolic risk factors, typical of individuals with impaired glucose tolerance or impaired fasting glycaemia, such as subjects with type II diabetes mellitus, prediabetes or metabolic syndrome, preferably in the regression of pre-diabetes conditions, such as impaired fasting glycaemia (IFG) conditions and/or impaired glucose tolerance (IGT) conditions.

The present composition further exhibited a synergistic effect with respect to the individual components.

Therefore, the object of the present invention is a composition comprising Citrus Sinensis (L.) Osbeck fruit extract and lemon extract and preferably further comprising at least one trace element, preferably a mineral, for example at least one mineral selected from the group consisting of: Chromium, Calcium, Magnesium, Iron, Copper, Zinc, Iodine, Manganese, Potassium, Sodium, Selenium, Molybdenum, Fluoride, Chloride or

Phosphorus or combinations thereof, and their salts and/or a vitamin, for example at least one vitamin selected from the group consisting of: A, D, E, K, B1, B2, C, Niacin, Pantothenic acid, B6, Folic acid, B12 or Biotin or combinations thereof.

Preferably the Citrus Sinensis (L.) Osbeck fruit is blood orange and/or blonde orange.

Preferably the composition of the invention comprises: Citrus sinensis (L.) Osbeck fruit extract, lemon extract and chromium, preferably in the form of a salt, preferably chromium picolinate. Preferably the composition of the invention comprises: blood orange and lemon extracts and chromium, preferably in the form of a salt, preferably chromium picolinate.

Preferably the composition of the invention comprises: blonde orange extract and lemon extract and chromium, preferably in the form of a salt, preferably chromium picolinate.

Preferably, the composition of the invention is characterized by a minimum flavonoid content equal to 48% by weight on the total weight of the composition and/or by a content equal to 0.5-5% by weight on the total weight of the composition, preferably about 0.8% w/w, in hydroxycinnamic acids. Preferably the flavonoids are present according to the following percentages by weight on the total weight of the composition: hesperidin 25-50%, preferably about 34%, eriocitrin 3-20%, preferably about 7%, anthocyanins 0.1-3%, preferably about 0.2%, other flavonoids including naringin, neoeriocitrin, neoesperidin, didymin, narirutin 3-10%, preferably about 7%.

Preferably in the composition of the invention flavonoids are present according to the following percentages by weight on the total weight of the composition: hesperidin about 34%, eriocitrin about 7%, anthocyanins about 0.2%, other flavonoids including naringin, neoeriocitrin, neoesperidin, didymin, narirutin about 7%.

The percentages indicated above are weight percentages on the total weight of the composition excluding the excipients.

The composition according to the invention is preferably for medical use. The composition according to the present invention is preferably for use in the prevention and/or treatment of alterations in glucose and/or glycolipid metabolism and/or a metabolic disorder, for example type II diabetes mellitus, prediabetes or metabolic syndrome, obesity, dyslipidemia and/or hyperglycemia and/or for use in reducing the cardio-metabolic risk factors, typical of individuals with impaired glucose tolerance or impaired fasting glycaemia, such as subjects with type II diabetes mellitus, prediabetes or metabolic syndrome, preferably in the regression of pre-diabetes conditions, such as impaired fasting glycaemia (IFG) conditions and/or impaired glucose tolerance (IGT) conditions.

Another object of the invention is a composition for oral administration containing as active ingredients at least the following flavonoid compounds: Anthocyanins, Hesperidin and Eriocitrin for use in the prevention and/or treatment of alterations in glucose and/or glycolipid metabolism and/or a metabolic disorder, for example type II diabetes mellitus, prediabetes or metabolic syndrome, obesity, dyslipidaemia and/or hyperglycemia and/or for use in reducing the cardio-metabolic risk factors, typical of individuals with impaired glucose tolerance or impaired fasting glycaemia, such as subjects with type II diabetes mellitus, prediabetes or metabolic syndrome, preferably in the regression of pre-diabetes conditions, such as impaired fasting glycaemia (IFG) conditions and/or impaired glucose tolerance (IGT) conditions.

The treatment of symptoms or disorders associated with the diseases listed above is also encompassed by the present invention.

Preferably, the composition according to the invention further comprises at least one trace element, preferably a mineral, for example at least one mineral selected from the group consisting of: Chromium, Calcium, Magnesium, Iron, Copper, Zinc, Iodine, Manganese, Potassium, Sodium, Selenium, Molybdenum, Fluoride, Chloride or Phosphorus or combinations thereof, and their salts and/or a vitamin, for example at least one vitamin selected from the group consisting of: A, D, E, K, B1, B2, C, Niacin, Pantothenic acid, B6, Folic acid, B12 or Biotin or combinations thereof, preferably a chromium salt, more preferably chromium picolinate.

The trace element is preferably chromium, preferably in the form of a salt, preferably it is chromium picolinate.

Preferably, the flavonoid compounds are extracted from pulp and/or peel of Citrus fruit, such as Citrus sinensis (L.) Osbeck fruit, preferably blood orange, and lemon.

Preferably, in the composition according to the invention:
a. the lemon extract is present in quantities from 100 to 1000 mg, preferably 250 mg, 500 mg or 1000 mg;
b. Citrus sinensis (L.) Osbeck fruit extract is present in quantities from 50 to 500 mg, preferably 100 mg or 150 mg, or 200 mg, or 400 mg.
c. the chromium is present in quantities from 20 mcg to 200 mcg, preferably 100 mcg, 200 mg or 50 mcg or 20 mcg.

Preferably, the composition according to the invention in unit dose form comprises: 100 to 1000 mg of lemon extract, preferably 250 mg or 500 mg or 1000 mg.

Preferably, the composition according to the invention in unit dose form comprises: from 50 to 500 mg of blood orange extract, preferably 100 mg, 150 mg, 200 mg, or 400 mg.

Preferably, the composition according to the invention in unit dose form comprises: from 20 mcg to 200 mcg, preferably 100 mcg, 200 mg or 50 mcg or 20 mcg.

Preferably, the composition or unit dose comprises:
lemon extract 500 mg
blood orange extract 200 mg
chromium 0.2 mg
or
lemon extract 250 mg
blood orange extract 100 mg
chromium 0.1 mg
or
lemon extract 1000 mg
blood orange extract 400 mg
chromium 0.2 mg
or
lemon extract 400 mg
blonde orange extract 50 mg
anthocyanins (from Vitis Vinifera fruit's peel extract) 7.5 mg
chromium 0.05 mg
or
lemon extract 250 mg
blonde orange extract 100 mg
anthocyanins (from Vaccinium myrtillus fruit extract) 150 mg
chromium 0.02 mg

Preferably chromium is in the form of a pharmaceutically acceptable salt, for example chromium chloride, chromium sulphate, chromium nitrate, chromium lactate trihydrate, chromium nicotinate, chromium picolinate, chromium citrate, or mixtures thereof. Preferably, chromium is chromium picolinate.

A further object of the invention is a pharmaceutical composition comprising the composition as defined above and at least one pharmaceutically acceptable excipient and/or carrier.

Another object of the invention is a food supplement or product or drinking product comprising the composition as described herein.

A further object of the invention is the non-therapeutic use of the composition as defined herein or of the food supplement or product or drinking product as defined herein in the nutraceutical sector or as a basic ingredient in supplement or drug preparations and/or as an agent for the prevention and/or treatment of alterations in glucose and/or glycolipid metabolism.

In the composition object of the present invention the extracts can be micronized to particles 0.1 to microns in diameter and/or incorporated in nanoparticles or in liposomal systems and/or 10 microencapsulated to increase the water solubility of the powders, the absorption and therefore the in vivo bioavailability of the polyphenol complex.

Trace elements such as vitamins and minerals can be added to such a composition, in addition to excipients.

Examples of vitamins preferably added to the present composition are: A, D, E, K, B1, B2, C, Niacin, Pantothenic acid, B6, Folic acid, B12 or Biotin or combinations thereof. These can be in the various possible vitamin formulas.

Examples of minerals preferably added to the present composition are: Calcium, Magnesium, Iron, Copper, Zinc, Iodine, Manganese, Potassium, Sodium, Selenium, Chromium, Molybdenum, Fluoride, Chloride or Phosphorus or combinations thereof. These can be in the form of the various possible mineral substances or pharmaceutically acceptable salts.

Preferably the composition comprises a pharmaceutically acceptable chromium salt, for example chromium chloride, chromium sulphate, chromium nitrate, chromium lactate trihydrate, chromium nicotinate, chromium picolinate, chromium citrate, or mixtures thereof. Preferably, said salt is chromium picolinate.

Preferably, the composition according to the invention comprises:
Citrus sinensis (L.) Osbeck fruit extract: % w/w from 5 to 50% and/or
lemon extract: % w/w from 5 to 70% and/or
chromium, preferably chromium picolinate: % w/w from 0.002% to 1%.

Preferably the composition of the invention further comprises anthocyanins, preferably from 1 mg to 300 mg, more preferably about 7 or 7.5 mg.

Said anthocyanins are comprised for example in Vitis Vinifera fruit extract or in Vitis Vinifera fruit peel extract or in Vaccinium myrtillus fruit extract. Therefore, the composition may comprise Vitis Vinifera fruit extract and/or Vitis Vinifera fruit peel extract and/or Vaccinium myrtillus fruit extract. In particular, when Citrus sinensis (L.) Osbeck fruit is blonde orange it is preferred that the composition comprises anthocyanins, as described above.

Preferably the composition of the invention is in solid, semi-solid or liquid form and/or is preferably in combination with one or more excipients selected from the group comprising: calcium phosphate, magnesium stearate, silicon dioxide, hydroxypropylcellulose, mono and diglycerides of fatty acids, microcrystalline cellulose, coating agents, maltodextrins, acidifiers of which preferably citric acid, preservatives of which preferably sodium benzoate and/or potassium sorbate, sweeteners of which preferably steviol glycosides, dyes of which preferably iron oxide; and/or in combination with other ingredients suitable for food or pharmaceutical use, such as one or more vitamins, minerals, enzymes, proteins, and/or other plant extracts.

Preferably in the composition according to the invention the extracts are micronized to particles 0.1 to 10 microns in diameter and/or incorporated in nanoparticles or in liposomal systems and/or microencapsulated to increase the water solubility of the powders, the absorption and therefore the in vivo bioavailability of the polyphenol complex.

The terms Citrus Sinensis (L.) Osbeck fruit extract and lemon extract (or blood orange extract and lemon extract) may be interchangeable with the expression Citrus Sinensis (L.) Osbeck fruit and lemon extracts (or blood orange and lemon extracts).

In the context of the present invention the expressions lemon extract and lemon fruit extract are interchangeable.

In the context of the present invention the expressions orange (or blood orange or blonde orange) extract and orange (or blood orange or blonde orange) fruit extract are interchangeable.

In the context of the present invention the expressions orange (or blood orange or blonde orange) extract and Citrus sinensis (L.) Osbeck fruit extract may be interchangeable.

The lemon extract and the Citrus sinensis (L.) Osbeck fruit extract may be obtained from the whole fruit or from any part of the fruit, e.g. from the juice and/or pulp and/or peel of the fruit or any combination thereof.

Preferably, the lemon extract is obtained from whole fruit (juice and/or pulp) and/or from the peel of the fruit.

The lemon or Citrus sinensis (L.) Osbeck fruit juice is preferably obtained by mechanical pressing of the fresh fruits.

The peels are preferably pressed and the juice obtained is mixed with the first obtained by pressing and the whole is preferably diluted with water. Subsequently, the semi-processed product is preferably subjected to an enzymatic treatment for the elimination of pectins and/or a filtration step, for example by passing the solution in ultrafiltration membranes with a molecular cut-off at 1000 Da. The filtrate is then preferably subjected to an extraction step by passage in specific adsorbent resins and the retentate is preferably subjected to a washing step with a mixture of water/ethanol. Finally, distillation is preferably performed for the recovery of the alcoholic solvent.

Preferably, the final product is dried for example by spray-drying.

The content in bioactive compounds of the extract, i.e., anthocyanins, flavanones (eriocitrin, hesperidin, narirutin, didymin) flavones (diosmin), hydroxycinnamic acids, ascorbic acid is calculated by HPLC technique (High performance liquid chromatography).

The extract can be standardized in the titre in bioactive compounds by appropriate addition of inert excipients in the final processing steps.

Preferably, said composition or pharmaceutical composition or food supplement is administered orally, preferably once or twice per day.

In the context of the present invention, the terms “preparation” or “composition” are used interchangeably.

The Citrus sinensis (L.) Osbeck fruit and lemon extract can be prepared by any method known to those skilled in the art, for example by mixing plant extracts obtained from different matrices: juice and/or pastazzo (residues of peel, pulp and seeds after pressing the fruit and eliminating the epicarp) and/or albedo by different extraction methods known to those skilled in the art (for example, resins and membranes, KOH, EtOH/H₂O/supercritical CO₂). Preferably the plant extract is or derives from juice, for example obtained by pressing ripe fruit, after eliminating the epicarp. The juice obtained by squeezing can be subjected to an enzymatic process for the elimination of the pectins. The liquid thus obtained is preferably subjected to a first membrane concentration process. The eluate is then preferably subjected to passage in adsorbent resins. The retentate of the resin is preferably washed with a mixture of water and ethanol and the liquid obtained is preferably subjected to solvent removal and a new membrane or heat concentration process. Lastly, the product is preferably dried by spray drying.

The lemon extract and Citrus sinensis (L.) Osbeck fruit extract are present in the composition according to the invention preferably in a weight ratio between: 5 to 1 and 1 to 5, preferably 2.5 to 1. The lemon and Citrus sinensis (L.) Osbeck fruit extracts and chromium are present in the composition according to the invention preferably in a weight ratio between: 5000 to 1 and 500 to 1. Preferably in a ratio of lemon extract and chromium of 2500 to 1 and of Citrus sinensis (L.) Osbeck fruit extract and chromium of 1000 to 1.

The lemon extract (here also called Citrus limon (L.) Osbeck. extract) according to the invention is preferably a dry, or dried, extract from the whole fruit (juice and pulp) and from the peel of the fruit. The blood orange extract (here also called orange or Citrus sinensis (L.) Osbeck extract or Citrus sinensis (L.) Osbeck fruit extract) according to the invention is preferably a dry, or dried, extract from the juice of the fruit and the peel.

Such dry powder extracts are commercially available and are obtained by hydroalcoholic extraction, preferably by extraction from ethanol/water and possibly filtration and concentration, addition of water, filtration and drying.

In the composition as described above, the components thereof are preferably dehydrated or dry or dried or in powder form.

A further object of the invention is a process for obtaining the composition as described above comprising adding chromium to the lemon and or Citrus sinensis (L.) Osbeck fruit extract, preferably chromium picolinate.

In the context of the present invention, the term comprising also includes the term consisting of or characterized by or consisting essentially of.

Said composition is preferably characterized in that said extracts or the composition itself is dehydrated or lyophilized or dry or dried or in the form of powder.

Still more preferably, said composition is characterized in that said extracts are dehydrated in the form of micronized, lyophilized or granulated particles.

The pharmaceutical composition according to the invention or the composition or food supplement according to the invention can be administered in the form of tablets, capsules, oral preparations, oral solutions, powders, granules, pills, injectables, or infusable liquid solutions, suspensions, suppositories, preparation for inhalation. Preferably, the total concentration of the extracts is between 10-90% by weight with respect to the total composition. They are normally presented in unit dose form and contain conventional excipients such as binders, fillers (comprising cellulose, mannitol, lactose), diluents, tablet agents, lubricants (including magnesium stearate), detergents, disintegrants (for example polyvinylpyrrolidone and starch derivatives such as sodium starch glycolate), colouring agents, flavouring agents and wetting agents (for example sodium lauryl sulphate).

The liquid preparations can be for example in aqueous form in oily suspension, solutions, emulsions, syrups or can be presented as a dry product for reconstitution with water. The liquid preparations can contain conventional additives, such as suspension agents, emulsifying agents, non-aqueous vehicles. A reference for the formulations is the book by Remington (“Remington: The Science and Practice of Pharmacy”, Lippincott Williams & Wilkins, 2000).

The composition, pharmaceutical composition or food supplement according to the invention can be administered in a single dosage containing all the components or as separate (simultaneous or sequential) compositions of the individual components. The composition, pharmaceutical composition or food supplement can be administered in combination with active ingredients which can be formulated separately in single-ingredient preparations of one of the forms described above and then administered as combined preparations which are given at the same time or at different times, or can be formulated together in the same preparation.

The person skilled in the art will choose the form of administration and the effective dosages, by selecting suitable diluents, adjuvants and/or excipients.

Preferably, said composition, pharmaceutical composition or food supplement is in solid form, for example a tablet, a hard capsule, a soft gel capsule, a powder, a syrup, a sachet, a lozenge, a tablet or a pill, small bag or stick pack, or in liquid form, for example an oral spray.

Preferably, said excipient or diluent or pharmaceutically acceptable vehicle is selected from the group consisting of: calcium phosphate, dicalcium phosphate, microcrystalline cellulose, magnesium stearate, silicon dioxide, sucrose, gum arabic, corn starch, medium chain triglycerides, tricalcium phosphate, cross-linked sodium carboxymethylcellulose, hydroxypropylmethylcellulose, polyethylene glycol, titanium dioxide, polyvinylpyrrolidone, talc, erythritol, xylitol, steviol glycosides and sucralose.

The pharmaceutical composition can be chosen based on the treatment to be performed. Preferably, said pharmaceutical composition is administered orally. Preferably, said pharmaceutical composition is administered once or twice per day.

The composition object of the present invention, preferably in the form of dry extracts, can be used in the nutraceutical field for example in the form of tablets by addition of at least one excipient, or it can be encapsulated in vegetable gelatin capsules with the addition of at least one pharmaceutically acceptable excipient.

The present composition can be mixed or combined with other active ingredients and/or substances of plant or natural or synthetic origin, and/or vitamins and/or minerals and/or with the addition of excipients or combinations thereof.

Examples of natural substances, plant extracts or fungi or derivatives thereof, or ingredients suitable for food or pharmaceutical use preferably present in the present composition are: alpha lipoic acid, acacia catechu, acacia decurrens, acacia farnesiana, acacia nilotica, acacia polycantha, acacia senegal, acacia seyal, ajuga iva, agaricus blazei, allium cepa, andrographis paniculata, anogeissus latifolia, anredera baselloides, artemisia dracunculus, artemisia pallens, berberis aquifolium, berberis aristata, bixa orellana, bowdichia virgilioides, cecropia peltata, chrysophyllum cainito, Cichorium intybus, cynara cardunculus, cynara scolymus, coscinium fenestratum, costus speciosus, cinnamomum verum, coriandrum sativum, cyclanthera and pedpeuter oleracea, exostema caribaeum, galega officinalis, gymnema sylvestre, hebante eriantha, hygrophila auriculata, hypomoea batatas, psyllium fibre, wheat or corn starch resistant dextrins, inulin, fructooligosaccharide, lagenaria siceraria, lapsana communis, lupinus spp, comerfera indica L., mamordica charantia, morinda officinalis, morus alba, morus nigra, ocimun tenuiflorum, monacolin k, berberine, Olea europaea, pachira aquatica, pachira insignis, panax ginseng, pelargonium radens, Phaseolus vulgaris, phyllantus amarus, phyllantus emblica, phyllantus niruri, plantago afra, plantago indica, planta ovata, punica granatum, rhodiola crenulata, glabra, ribes nigrum, ribes rubrum, ribes uva-crispa, rubus fruticosus, rubus idaeus, rubus chingii, sarcopoterium, sambucus nigra, sambucus canadensis, sambucus ebulus, silibum marianum, sygium, syzygium jambos, syzygium malaccense, trigonella foenum-graecum, Triticum aestivum, vanilla planifolia, yucca filamentosa, yucca schidigera, policosanols, plant sterols or stanols, silymarin, green tea extracts, vaccinium corymbosum, vaccinium macrocarpon, vaccinium myrtilloides, vaccinium myrtillus, vaccinium oxycoccos Vitis vinifera, monounsaturated or polyunsaturated fatty acids or chitosan or combinations thereof.

Examples of vitamins preferably present in the present composition are: A, D, E, K, B1, B2, C, Niacin, Pantothenic acid, B6, Folic acid, B12 or Biotin or combinations thereof. These can be in the various possible vitamin formulas.

Examples of minerals preferably present in the present composition are: Calcium, Magnesium, Iron,

Copper, Zinc, Iodine, Manganese, Potassium, Sodium, Selenium, Chromium, Molybdenum, Fluoride, Chloride or Phosphorus or combinations thereof. These can be in the form of the various possible mineral substances.

A further object of the present invention is a food supplement or a food product or a drinking product comprising the composition as described above and at least one excipient or diluent and, optionally, a further agent.

Preferably, said excipient and/or diluent is selected from the group consisting of: calcium phosphate, dicalcium phosphate, calcium carbonate, microcrystalline cellulose, magnesium stearate, silicon dioxide, sucrose, gum arabic, corn starch, medium chain triglycerides, tricalcium phosphate, cross-linked sodium carboxymethylcellulose, hydroxypropylmethylcellulose, polyethylene glycol, titanium dioxide, polyvinylpyrrolidone, talc, erythritol, xylitol, steviol glycosides and sucralose. Preferably, said food supplement or product or drinking product is administered orally. Preferably, said food supplement or product or drinking product is administered once or twice per day. Preferably, said food supplement or product or drinking product is in the form of a tablet, a hard capsule, a soft gel capsule, a powder, a syrup, a cachet, a lozenge, a tablet, a lozenge, a food supplement, an edible bar, or an edible snack. In particular, said supplement may be any type of food supplement.

The preferred aspects indicated above for the individual components are also to be considered preferred in the unit dose embodiments.

The invention will be illustrated by non-limiting examples.

### EXAMPLES

### Example 1: Preparation Example of a Lemon Extract to be Integrated in the Composition According to the Invention

The botanical name of the lemon extract used is Citrus limon (L.) Osbeck. Such an extract is obtained from the whole fruit (juice and pulp) and from the peel of the fruit. The juice of Citrus limon (L.) Osbeck is obtained by mechanical pressing of the fresh fruits, well ripe, free of rot and mould.

The peels are pressed and the juice obtained is mixed with the first obtained by pressing and the whole diluted with water. Subsequently, the semi-processed product is subjected to an enzymatic treatment for the elimination of pectins and to a filtration step by passing the solution in ultrafiltration membranes with a molecular cut-off at 1000 Da. The extraction step includes the passage in specific adsorbent resins and the retentate is subjected to a washing step with water/ethanol mixture. Distillation follows to recover the alcoholic solvent.

The final product is finally subjected to a spray-drying step obtaining a straw yellow powder with an intense smell of citrus.

The HPLC (High Performance Liquid Chromatography) analysis shows the content of bioactive compounds of the extract, namely flavanones (eriocitrin, hesperidin, narirutin) flavones (diosmin), hydroxycinnamic acids, ascorbic acid.

The extract can be standardized in the titre in bioactive compounds by appropriate addition of inert excipients in the final processing steps.

### Example 2: Preparation Example of Blood Orange or Blonde Orange Extract to be Integrated in the Composition According to the Invention

The botanical name of the blood orange extract used is Citrus sinensis (L.) Osbeck. Such an extract is obtained from the juice of the fruit and from the peel. The juice of Citrus sinensis (L.) Osbeck is obtained by mechanical pressing of the fresh fruits, well ripe, free of rot and mould.

The peels are pressed and the juice obtained is mixed with the first obtained by pressing and the whole diluted with water. Subsequently, the semi-processed product is subjected to an enzymatic treatment for the elimination of pectins and to a filtration step by passing the solution in ultrafiltration membranes with a molecular cut-off at 1000 Da. The extraction step includes the passage in specific adsorbent resins and the retentate is subjected to a washing step with water/ethanol mixture. Distillation follows to recover the alcoholic solvent.

The final product is finally subjected to a spray-drying step obtaining a bright pink/red powder with an intense smell of citrus.

The extract is then titrated using HPLC (high performance liquid chromatography). The analysis shows the content in bioactive compounds of the extract, namely anthocyanins, flavanones (hesperidin, narirutin, didymin) flavones (diosmin), hydroxycinnamic acids.

The extract can be standardized in the titre in bioactive compounds by appropriate addition of inert excipients in the final processing steps.

Blonde orange extract may be prepared in a similar way.

### Example 3: Example of Composition According to the Invention in the Form of Swallowable Tablets

The composition object of the present invention is in the form of swallowable tablets. It comprises the following ingredients, in addition to excipients such as calcium phosphate, magnesium stearate, silicon dioxide, hydroxypropylcellulose, mono- and diglycerides of fatty acids, microcrystalline cellulose:

| | Quantity per 1 | Nutritional reference values |
|---|---|---|
| List of ingredients | tablet (mg) | for 1 tablet |
| Lemon extract | 250 | — |
| Blood orange extract | 100 | — |
| Total polyphenols | 174 | — |
| of which | 3.0 | |
| hydroxycinnamic acids | | |
| of which flavonoids: | 171 | |
| Anthocyanins | 1 | |
| Hesperidin | 120 | |
| Eriocitrin | 25 | |
| other flavonoids | 25 | |
| Chromium | 0.1 | 250% |

* Nutritional reference values as per Reg EU 1169/2011

### Example 4: Example of Composition According to the Invention in the Form of a Powder in Sachet

The composition object of the present invention is in the form of a powder in sachet to be dissolved in water.

It contains the following ingredients in addition to excipients such as maltodextrins, silicon dioxide, steviol glycosides as a sweetener:

| | Quantity per 1 | Nutritional reference values |
|---|---|---|
| List of ingredients | sachet (mg) | for 1 sachet |
| Lemon extract | 500 | — |
| Blood orange extract | 200 | — |
| Total polyphenols | 348 | — |
| of which | 6.0 | |
| hydroxycinnamic acids | | |
| of which flavonoids: | 342 | |
| Anthocyanins | 2 | |
| Hesperidin | 240 | |
| Eriocitrin | 50 | |
| other flavonoids | 50 | |
| Chromium | 0.2 | 500% |

* Nutritional reference values as per Reg EU 1169/2011

### Example 5: Example of Composition According to the Invention in Liquid Form in Vial

The composition object of the invention is presented in the form of a beverage in a vial, containing the following ingredients, as a percentage of the total weight of the composition, per single dose: lemon extract 10%, blood orange extract 20%, chromium 0.002% in addition to excipients such as water, thickeners, sweeteners, dyes, citric acid as an acidifier, sodium benzoate and potassium sorbate as preservatives.

| | Quantity per 1 | Nutritional reference values* |
|---|---|---|
| List of ingredients | vial (mg) | for 1 vial |
| Lemon extract | 1000 | — |
| Blood orange extract | 400 | — |
| Total polyphenols | 696 | — |
| of which | 12 | |
| hydroxycinnamic acids | | |
| of which flavonoids: | 684 | |
| Anthocyanins | 4 | |
| Hesperidin | 480 | |
| Eriocitrin | 100 | |
| other flavonoids | 100 | |
| Chromium | 0.2 | 500% |

*Nutritional reference values as per Reg EU 1169/2011

### Example 6: Example Composition According to the Invention in the Form of Swallowable Capsules

The composition object of the present invention is in the form of swallowable capsules. It comprises the following ingredients, in addition to excipients such as hydroxypropyl methylcellulose, maltodextrins, magnesium salts of fatty acids, silicon dioxide.

| | Quantity per 1 | Nutritional reference values |
|---|---|---|
| List of ingredients | tablet (mg) | for 1 tablet |
| Lemon extract | 500 | — |
| Blond orange extract | 150 | — |
| Total polyphenols | 255 | — |
| of which | 5.0 | |
| hydroxycinnamic acids | | |
| of which flavonoids: | 250 | |
| Anthocyanins | 100 | |
| Hesperidin | 120 | |
| Eriocitrin | 25 | |
| other flavonoids | 5 | |
| Chromium | 0.1 | 250% |

* Nutritional reference values as per Reg EU 1169/2011

### Example 7: Clinical Evaluation of the Efficacy of the Composition

A single-centre, 4-arm, double-blind, placebo-controlled, pilot clinical study was conducted to evaluate the efficacy of the composition object of the present invention on humans in reducing insulin resistance-related risk factors. The study was carried out in accordance with the guidelines of the Helsinki Declaration and approved by the Ethics Committee of the Hospital. 100 adult individuals of both sexes were enrolled (47 women and 53 men, aged 20 to 75 years) with dysglycaemia (baseline blood glucose between 100 and 125 mg/dL on at least two consecutive occasions), in primary prevention for cardiovascular disease.

Exclusion criteria: dysglycaemia secondary to pathological or iatrogenic causes, systemic diseases associated with dysglycaemia, uncompensated dysthyroidism, treatment with drugs potentially interacting with glucose metabolism (for example corticosteroids, atypical antipsychotics).

The subjects enrolled were randomized into 5 groups, each consisting of 20 individuals:
Group A: 1 tablet of active (COMP) twice daily
Group B: 1 placebo tablet twice daily
Group C: 1 tablet of lemon extract 250 mg twice daily
Group D: 1 tablet of blood orange extract 100 mg twice daily
Group E: 1 tablet of chromium 100 mcg twice daily

All the enrolled subjects were subjected, 14 days prior to enrolment (T-14), to a pre-enrolment visit with blood tests to assess meeting the inclusion criteria. The individuals who met the TO inclusion criteria underwent a randomization visit at which the respective treatment was assigned and blood tests were performed in relation to the reference parameters.

The study lasted 12 weeks.

At the end of treatment (T12), the subjects were subjected to an end-of-study visit and blood tests.

The parameters investigated in the study are: Body mass index, systolic and diastolic blood pressure, glycated haemoglobin (HbAlc) fasting plasma glucose (FPG), fasting insulinemia (FPI), HOMA-index, triglycerides, gammaGT, creatinine phosphokinase (CPK).

The treatment called “active” or “COMPOSITION (COMP)” consists of a 570 mg tablet consisting of 250 mg lemon extract, 100 mg blood orange extract, 100 mcg chromium (as chromium picolinate), in addition to excipients such as calcium phosphate, magnesium stearate, silicon dioxide, hydroxypropyl cellulose, mono- and di-glycerides of fatty acids, microcrystalline cellulose, yellow iron oxide as a dye, coating agents. The treatment called “placebo” consists of a tablet equal in shape, weight, colour size and flavour to the active tablet but containing “inert” material for the desired effect and consists of: microcrystalline cellulose, magnesium stearate, silicon dioxide, calcium phosphate, chlorophylline dye, yellow iron oxide dye, coating agents.

The treatments related to groups C, D and E consist of one tablet equal in appearance to the tablets of groups A and B but consisting of the single active ingredient, as described above in addition to excipients.

The treatment tablets were packaged in anonymous white bottles, showing only the batch number consisting of an alphanumeric code and the expiration date. Neither the investigator nor the study subjects knew the contents of the bottles. The bottles of each treatment were placed in cardboard boxes containing a letter in a sealed envelope with reference to the batch number and content, to be opened only in case of need, as in cases of serious adverse events. After randomization, each study participant was given the bottle with 60 tablets for the specific treatment of the group to which they belonged.

All the subjects were instructed to take one tablet in the morning and one in the evening for 12 weeks. The results are described in table 1.

After 12 weeks of treatment, group A achieved statistically significant reductions with respect to both the baseline and placebo in the following parameters: HbAlc, FPG, FPI, HOMA-index, triglycerides.

The groups B, C, D, E did not show statistically significant reductions in the parameters of interest. There were no significant changes in systolic and diastolic blood pressure, body mass index, creatine phosphokinase (CPK), gammaGT, for any of the treatment groups.

In particular, the results of group A were significantly higher with respect to the individual groups C, D and E and higher with respect to the sum of the individual results of groups C, D, E, showing a synergistic effect of the composition object of the present invention.

Regarding the results related to glycated haemoglobin (HbAlc), group A achieved a reduction of 10.7% while group B, group C, group D, group E did not achieve reductions.

With regard to fasting plasma glucose (FPG) results, group A achieved a reduction of 28%, group B of 0.8%, group C of 8.4%, group D of 8.3%, group E of 4.2%. The sum of the reductions of groups C,D and E is therefore equal to 20.9% lower than the result obtained with the composition object of the invention.

With regard to fasting insulinemia (FPI), group A achieved a reduction of 34.7%, group B 1.5%, group C 8%, group D 12% and group E 4%. The sum of the reductions of the individual active ingredients is therefore equal to 24% less than the result obtained with the composition object of the invention.

In the variations of the HOMA index, calculated according to the formula HOMA-IR=(FPG*FPI)/405, group A showed a 52.5% reduction, group B 2.6%, group C 15%, group D 20%, group E 13%. The sum is 48%, lower than the result obtained with the composition object of the invention.

In the changes in triglycerides (TG), group A showed a statistically significant reduction of 30%, therefore higher than the sum of the reductions in the treatment groups with the individual substances, equal to −10.3%.

TABLE 1
Changes in the parameters investigated during the study.

| | GROUP A | | GROUP B | | GROUP C | | GROUP C | | GROUP C | |
|---|---|---|---|---|---|---|---|---|---|---|
| | (COMP) | | (PLACEBO) | | (LEMON) | | (BLOOD | | (CHROMIUM) | |
| | (n = 20; | | (n = 20; | | (n = 20; | | ORANGE) (n = | | (n = 20; | |
| | F = 10, M = 10) | | F = 9, M = 11) | | F = 9, M = 11) | | 20; F = 8, M = 12) | | F = 11, M = 9) | |
| | | 12- | | 12- | | 12- | | 12- | | 12- |
| | | Week | | Week | | Week | | Week | | Week |
| | | follow- | | follow- | | follow- | | follow- | | follow- |
| | Baseline | up | Baseline | up | Baseline | up | Baseline | up | Baseline | up |
| BMI | 26.0 ± 1.0 | 26.0 ± 0.8 | 26.0 ± 0.9 | 26.0 ± 1.0 | 25.9 ± 1.0 | 25.9 ± 1.0 | 25.8 ± 0.8 | 25.8 ± 1.0 | 26.2 ± 1.0 | 26.2 ± 1.0 |
| (kg/m2) | | | | | | | | | | |
| SBP | 131 ± 7 | 130 ± 7 | 128 ± 8 | 126 ± 6 | 126 ± 9 | 124 ± 7 | 130 ± 7 | 129 ± 6 | 131 ± 5 | 130 ± 7 |
| (mmHg) | | | | | | | | | | |
| DBP | 84 ± 5 | 83 ± 6 | 85 ± 6 | 84 ± 5 | 84 ± 7 | 83 ± 6 | 84 ± 6 | 83 ± 7 | 85 ± 6 | 84 ± 4 |
| (mmHg) | | | | | | | | | | |
| FPG | 125 ± 9 | 90 ± 6*° | 119 ± 6 | 118 ± 9 | 118 ± 8 | 108 ± 5 | 120 ± 6 | 110 ± 8 | 117 ± 5 | 112 ± 4 |
| (mg/dL) | | | | | | | | | | |
| FPI | 25.3 ± 1.6 | 16.5 ± 1.13*° | 25.6 ± 1.9 | 25.2 ± 1.1 | 25.0 ± 1.2 | 23.0 ± 1.7 | 25.5 ± 1.9 | 22.3 ± 1.2 | 22.9 ± 1.0 | 20.9 ± 1.1 |
| (μU/ml) | | | | | | | | | | |
| TG | 165 ± 11 | 115 ± 12*° | 159 ± 12 | 160 ± 10 | 151 ± 13 | 148 ± 12 | 151 ± 13 | 145 ± 9 | 159 ± 12 | 152 ± 10 |
| (mg/dL) | | | | | | | | | | |
| HbA1c | 6.4 ± 0.2 | 5.7 ± 0.1*° | 6.3 ± 0.3 | 6.3 ± 0.2 | 6.4 ± 0.3 | 6.4 ± 0.1 | 6.2 ± 0.2 | 6.2 ± 0.3 | 6.4 ± 0.3 | 6.4 ± 0.1 |
| (%) | | | | | | | | | | |
| HOMA- | 7.8 ± 1.6 | 3.7 ± 1.2*° | 7.5 ± 1.8 | 7.3 ± 1.7 | 7.2 ± 1.5 | 6.1 ± 1.0 | 7.5 ± 1.1 | 6.0 ± 1.6 | 6.6 ± 1.2 | 5.7 ± 1.8 |
| IR | | | | | | | | | | |
| Gamma- | 28 ± 9 | 25 ± 9 | 25 ± 7 | 24 ± 6 | 26 ± 9 | 23 ± 6 | 25 ± 6 | 24 ± 5 | 24 ± 9 | 27 ± 6 |
| GT (U/L) | | | | | | | | | | |
| CPK | 121 ± 23 | 127 ± 38 | 134 ± 42 | 130 ± 28 | 129 ± 37 | 131 ± 21 | 132 ± 22 | 1131 ± 27 | 134 ± 25 | 133 ± 22 |
| (U/L) | | | | | | | | | | |

*P < 0.05 vs. baseline;
°P < 0.05 vs. placebo.

### Example 8

A study was conducted in order to assess the efficacy of the composition of the present invention versus a composition consisting of extracts of lemon and red orange.

20 individuals with fasting plasma glucose (FPG) between 100 and 125 mg/dL (in two consecutive occasions) were enrolled and randomized in 2 groups.

Individuals of Group 1 (N=10) were instructed to take, twice daily after main meals, a composition in form of swallowable tablets consisting of 250 mg of lemon fruit extract and 100 mg of orange fruit extract for 12 weeks (COMP 1).

Individuals of Group 2 (N=10) took, twice daily for 12 weeks, a composition, in the form of swallowable tablets, consisting of 48% by weight on the total weight of the composition excluding the excipients of flavonoids from Citrus sinensis (L.) Osbeck, such as blonde orange, and Citrus Limon L. Osbeck (Hesperidin, Ericitrin), 7.5 mg anthocyanins (such as from Vitis Vinifera fruit extract) (Cyanidin, Malvidin, Delphinidin, Petunidin) plus 100 mcg of Chromium picolinate (COMP 2).

Results in FPG at the baseline and after 12 weeks show a marked reduction of FPG in group 2 with respect to group 1 (p<0.001)

These results show an unexpected increased effect of the composition object of the invention with respect of a composition comprising only extracts from orange and lemon fruits.

In fact after 12 weeks Group 1 achieved an FPG reduction of −19.2% and Group 2 of −31%.

Comparing these results with what obtained with Chromium alone (Group E of example 7) it seems clear that the combination of Flavonoids from Citrus sinensis and citrus limon, including anthocyanins and Chromium picolinate has a synergistic effect.

In order to confirm the synergistic effect the method described by S. R. Colby in “Calculation of the synergistic and antagonistic responses of herbicide combinations” Weeds, 1967 was applied.

The synergy factor was calculated for each compound. A factor >1 indicates the existence of a synergistic effect. A factor <1 indicates the existence of an antagonistic effect.

The formula applied in the calculations is as follows:

$$Expected efficacy rate = A + B - ( A * B / 100 )$$

$$Synerg y factor ( FS ) = ( 1 * observed efficacy rate ( \% ) ) / expected efficacy rate ( \% )$$

Calculation of the synergy factor for A+B=C where A=COMP 1 (composition of orange and lemon fruit extracts), B=Chromium picolinate 100 mcg and C═COMP 2 (composition of lemon and orange fruit extracts and Chromium)

| | Observed efficacy | | | Expected | |
|---|---|---|---|---|---|
| | rate (%) | | | efficacy | Synergy |
| | A | B | C | rate for C | factor |
| Reduction of FPG (−) | −19.2 | −4.2 | −31 | −22.6% | 1.37 |

The synergy factor value (1,37) confirms that there is a synergistic effect of COMP 2 versus the combination of orange and lemon extract and chromium alone.

Results similar to those obtained with the above COMP 2 were obtained by administering a composition, in the form of swallowable tablets, comprising 350 mg of fruit extracts of blood orange and of lemon (ratio 1:2,5) plus 100 mcg of Chromium picolinate (COMP 3).

1. A composition comprising Citrus sinensis (L.) Osbeck fruit extract and lemon extract
and optionally further comprising at least one trace element and/or a vitamin.

2. The composition according to claim 1 comprising: Citrus sinensis (L.) Osbeck fruit extract, lemon extract and chromium, wherein the chromium is optionally in the form of a salt, or is chromium picolinate.

3. The composition according to claim 1 comprising: blood orange extract and lemon extract and chromium, wherein the chromium is optionally in the form of a salt, or is chromium picolinate.

4. The composition according to claim 1 comprising: blonde orange extract and lemon extract and chromium wherein the chromium is optionally in the form of a salt, or is chromium picolinate.

5. The composition according to claim 1, a wherein the minimum flavonoid content is equal to 48% by weight based on the total weight of the composition and the content of flavonoid hydroxycinnamic acids is equal to 0.8% w/w.

6. The composition according to claim 1, wherein flavonoids are present according to the following percentages by weight based on the total weight of the composition: hesperidin about 34%, eriocitrin about 7%, anthocyanins about 0.2%, other flavonoids including naringin, neoeriocitrin, neoesperidin, didymin, narirutin about 7%.

7. A method for the prevention and/or treatment of alterations in glucose and/or glycolipid metabolism and/or of a metabolic disorder and/or for use in reducing cardio-metabolic risk factors, typical of individuals with impaired glucose tolerance or with impaired fasting glucose, comprising administering a composition of claim 1 to a subject in need of such treatment or prevention.

8. The method of claim 7, wherein said composition contains as active ingredients at least the following flavonoid compounds: Anthocyanins, Hesperidin and Eriocitrin.

9. The method according to claim 8, wherein said composition further comprises a trace element, said trace element optionally being a mineral or a vitamin.

10. The method according to claim 8 wherein the trace element is chromium, optionally in the form of a salt, or as chromium picolinate.

11. The method according to claim 8 wherein the flavonoid compounds are extracted from pulp and/or peel of citrus fruits, such as blood orange and lemon.

12. The method according to claim 1, wherein:
a. lemon extract is present in quantities from 100 to 1000 mg;
b. Citrus sinensis (L.) Osbeck fruit extract is present in quantities from 50 to 500 mg, and
c. chromium is present in quantities from 20 mcg or from 100 mcg to 200 mcg.

13. Pharmaceutical composition comprising the composition as defined in claim 1 and at least one pharmaceutically acceptable excipient and/or carrier.

14. A food supplement or product or drink product comprising the composition of claim 1.

15. (canceled)

16. The composition of claim 1, wherein said one trace element is a mineral, and said mineral is optionally selected from the group consisting of: Chromium, Calcium, Magnesium, Iron, Copper, Zinc, Iodine, Manganese, Potassium, Sodium, Selenium, Molybdenum, Fluoride, Chloride or Phosphorus or combinations thereof, and their salts; and wherein said vitamin is selected from the group consisting of: A, D, E, K, B1, B2, C, Niacin, Pantothenic acid, B6, Folic acid, B12 or Biotin or combinations thereof.
